(19) United States
(12) Patent Application Publication
(10) Pub. No.: US 2021/0321885 A1
(43) Pub. Date: Oct. 21, 2021

(54) BIO SHIELD FOR DENTAL CHAIR

(71) Applicant: ZYBERGRAPH LLC, Billings, MT (US)

(72) Inventors: Spencer E. ZAUGG, Billings, MT (US); Colton L. ZAUGG, Billings, MT (US); Marlin D. RICHARDSON, Bililngs, MT (US)

(73) Assignee: ZYBERGRAPH LLC, Bililngs, MT (US)

(21) Appl. No.: 17/236,747

(22) Filed: Apr. 21, 2021

Related U.S. Application Data
(60) Provisional application No. 63/013,464, filed on Apr. 21, 2020.

(51) Int. Cl. A61B 5/0205; A61B 90/40; A61B 5/00; A61B 5/145; A61B 5/1468; A61B 90/25; A61B 90/35

(52) U.S. Cl. CPC A61B 5/02055; A61B 90/40; A61B 5/7405; A61B 5/7425; A61B 5/748; A61B 2090/401; A61B 5/14542; A61B 5/1468; A61B 90/25; A61B 90/35; A61B 5/0077; A61B 5/0022

(57) ABSTRACT

A shield device and related methods of use for conducting a patient procedure, such as a dental procedure, when the patient is positioned in or on a chair or table. The shield device includes a support arm and a shield. The shield is mounted to the support arm and movable between an active position vertically above a portion of the patient when the patient is lying in the chair or table, and an inactive position removed from patient. The shield has a transparent portion through which a user can view the patient when the shield is in the active position. The shield includes a ventilation system to remove air from a space defined between the patient and the shield when the shield is in the active position.

### CROSS REFERENCE TO RELATED APPLICATIONS

This application claims priority to U.S. Patent Application No. 63/013,464 entitled “BIO SHIELD FOR DENTAL CHAIR,” filed Apr. 21, 2020 which is incorporated herein in its entirety by this reference.

### TECHNICAL FIELD

The present disclosure relates generally to biological shields, and more particularly to biological shields for use in dental office settings.

### BACKGROUND

In a number of industries, such as medical, dentistry, veterinary and laboratory, the use of a face shield provides protection for the eyes and face of the wearer from debris, splatters or pathogens. Increasingly, face shields are being utilized for preventing infections that can occur as a result of bodily fluid splattering that can arise within a number of occupations. A number of factors affect the acceptance of a face shield design, including comfort, ventilation, weight, ability to be securely retained in position, cost, and aesthetic considerations. The need for secure positioning and ventilation, which increases comfort while reducing the possibility of fogging, can be critical performance factors, especially in situations in which the face shield needs to be worn for extended periods of time.

Additionally, glare control is an important consideration for medical personnel working under bright lights, outdoors or who wear glasses under the face shield. Glare causes eye fatigue and facial fatigue from squinting and can contribute to loss of productivity or errors over an extended period of time. In certain light conditions, parts of eyeglasses worn by the user are reflected on the inside of a face shield thereby interfering with vision through the face shield.

Medical face shields are used to prevent transfer of pathogens and, during use, can become contaminated by these pathogens. Therefore, disposable face shields are highly desired. To be practical, disposable face shields should be low cost and easy to use by the wearer. Since face shield assemblies are typically a three dimensional shape when worn, a kit that allows face shield components to be packaged and shipped in a compact, flat configuration and assembled at the point of use is desirable.

A number of examples of practical face shields exist. It will be appreciated, however, that the growing market for face shields is always in search of improved face shield designs that provide increasing utility, comfort, and style while reducing material requirements and manufacturing costs.

### SUMMARY

One aspect of the present disclosure is directed to a shield device for use with conducting a patient procedure, such as a dental procedure, when the patient is positioned in or on a chair or table. The shield device includes a support arm and a shield. The shield is mounted to the support arm and movable between an active position vertically above a portion of the patient when the patient is lying in the chair or table, and an inactive position removed from patient. The shield has a transparent portion through which a user can view the patient when the shield is in the active position. The shield includes a ventilation system to remove air from a space defined between the patient and the shield when the shield is in the active position.

The ventilation system may include at least one vent opening positioned on a side of the shield facing the patient. The shield may include a frame member surrounding the transparent portion, and the ventilation system may include a plurality of vent openings formed in the frame member on a side of the shield facing the patient. The shield device may include a skirt connected to the shield and extending toward the patient. The shield device may include at least one light source mounted to the shield and directed toward the patient. The shield device may include a speaker mounted to the shield. The shield device may include a display mounted to the shield and directed toward the patient for viewing by the patient. The shield may include a touch screen configured to receive touch inputs from the user. The shield may include a magnification portion, or a portion that have an adjustable magnification feature.

The shield device may include a user display mounted to the shield and configured to display at least one visual indicator or message. The user display may display patient vital signs in real time. The user display may display at least one of the patient's dental history, x-rays of the patient's teeth, current time, and recommended instructions for a procedure. The shield device may include at least one communications port, and the communications port may be configured to connect a mobile communications device to the shield. The shield device may include at least one camera mounted to the shield, and the camera may be directed toward the patient or toward the user, or include separate cameras directed toward the patient and user. The shield device may include a disposable cover extending over at least one surface of the shield. The transparent portion of the shield may include at least one of tempered glass and acrylic material. The shield device may include at least one sensor to detect one or more of temperature, light, eye position of the user, sound frequency, sound volume, smoke, oxygen saturation, and chemical compounds.

The support arm may be mounted to a chair in which the patient is sitting. The support arm may be mounted to a ceiling at a location vertically above the patient. The support arm may be mounted to a wall positioned adjacent to the patient. The support arm may be mounted to a floor or supported by a floor. The support arm may include a plurality of arm segments to adjustably position the shield at a desired location relative to the patient. The support arm may include a plurality of pivot joints coupling together a plurality of arm segments, and a controller to move the arm segments automatically in response to user controls. The user controls may include at least one of a touch input or an audible input.

The shield device may include a laser filter mounted to the shield and configured to filter at least one of CO2 laser light, Diode laser light, and yttrium aluminum garnet (YAG) laser light. The shield device may include a microscope mount and be configured to releasably mount a microscope for viewing the patient or procedure site. A portion of the ventilation system may extend within or along the support arm.

Another aspect of the present disclosure relates to a shield device for use with a dental chair. The shield device includes a support arm, and a shield mounted to the support arm and configured to be positioned above a patient's head when the patient is lying in the dental chair. The shield includes a frame to which the support arm is mounted, a transparent portion mounted to the frame, the patient's head being viewable through the transparent portion, and a ventilation system configured to remove air from a space defined between the patient's head and the shield. At least a portion of the ventilation system is defined by or supported by the frame.

The foregoing has outlined rather broadly the features and technical advantages of examples according to the disclosure in order that the detailed description that follows may be better understood. Additional features and advantages will be described hereinafter. The conception and specific examples disclosed may be readily utilized as a basis for modifying or designing other structures for carrying out the same purposes of the present disclosure. Such equivalent constructions do not depart from the spirit and scope of the appended claims. Features which are believed to be characteristic of the concepts disclosed herein, both as to their organization and method of operation, together with associated advantages will be better understood from the following description when considered in connection with the accompanying figures. Each of the figures is provided for the purpose of illustration and description only, and not as a definition of the limits of the claims.

### BRIEF DESCRIPTION OF THE DRAWINGS

A further understanding of the nature and advantages of the embodiments may be realized by reference to the following drawings. In the appended figures, similar components or features may have the same reference label.

FIG. 1 is a perspective view of an example shield device in use between a user such as a doctor and a patient in accordance with the principals of the present disclosure.

FIG. 2 is a side view of an example shield device supported on a ground surface in accordance with the present disclosure.

FIG. 3 is a side view of another example shield device mounted to a wall surface in accordance with the present disclosure.

FIG. 4 is a side view of another example shield device mounted to a portable stand in accordance with the present disclosure.

FIG. 5 is a side view of an example shield device mounted to a frame of a patient chair in accordance with the present disclosure.

FIG. 6 is a side view of an example shield device mounted to a base support of a patient chair in accordance with the present disclosure.

FIG. 7 is a side view of an example shield device mounted to a ceiling above a patient chair in a first configuration in accordance with the present disclosure.

FIG. 8 is a side view of the shield device of FIG. 7 in a second configuration in accordance with the present disclosure.

FIG. 9 is a side view of an example shield device mounted to a wall in accordance with the present disclosure.

FIG. 10 is a side view of an example shield device mounted to a floor in accordance with the present disclosure.

FIGS. 11A and 11B show a mounting bracket for use with the shield devices disclosed herein.

FIG. 12 is a bottom view of an example shield in accordance with the present disclosure.

FIG. 13 is a top view of an example shield with a magnifying portion in accordance with the present disclosure.

FIG. 14 is a top view of an example shield having a display portion in accordance with the present disclosure.

FIG. 15 is a top view of an example shield having a display portion in accordance with the present disclosure.

FIG. 16 is a bottom view of an example shield device in accordance with the present disclosure.

FIG. 17 is a top view of an example shield device in accordance with the present disclosure.

FIG. 18 is a bottom view of an example shield in accordance with the present disclosure.

FIG. 19 is a top view of an example shield in accordance with the present disclosure.

FIG. 20 is a top view of an example shield having a skirt in accordance with the present disclosure.

FIG. 21 is a top view of an example shield device having a skirt and mounted to a patient chair in accordance with the present disclosure.

FIG. 22 is a side view of the shield device shown in FIG. 22.

FIG. 23 is a rear perspective view of the shield device shown in FIG. 21.

FIG. 24 is a rear perspective view of the shield device shown in FIG. 21 with the skirt removed.

FIG. 25 is a top perspective view of the shield device shown in FIG. 21 with the skirt removed.

FIG. 26 shows a diagram of a system including one or more devices for control of aspects of a shield device in accordance with the present disclosure.

While the embodiments described herein are susceptible to various modifications and alternative forms, specific embodiments have been shown by way of example in the drawings and will be described in detail herein. However, the exemplary embodiments described herein are not intended to be limited to the particular forms disclosed. Rather, the instant disclosure covers all modifications, equivalents, and alternatives falling within the scope of the appended claims.

### DETAILED DESCRIPTION

This description provides examples, and is not intended to limit the scope, applicability or configuration of the invention. Rather, the ensuing description will provide those skilled in the art with an enabling description for implementing embodiments of the invention. Various changes may be made in the function and arrangement of elements.

Thus, various embodiments may omit, substitute, or add various procedures or components as appropriate. For instance, it should be appreciated that the methods may be performed in an order different than that described, and that various steps may be added, omitted or combined. Also, aspects and elements described with respect to certain embodiments may be combined in various other embodiments. It should also be appreciated that the following systems, methods, and devices may individually or collectively be components of a larger system, wherein other procedures may take precedence over or otherwise modify their application.

The detailed description of exemplary embodiments herein makes reference to the accompanying drawings, which show exemplary embodiments by way of illustration. While these exemplary embodiments are described in sufficient detail to enable those skilled in the art to practice the disclosure, it should be understood that other embodiments may be realized and that logical changes and adaptations in design and construction may be made in accordance with this disclosure and the teachings herein without departing from the spirit and scope of the disclosure. Thus, the detailed description herein is presented for purposes of illustration only and not of limitation.

In various embodiments, and with reference to the accompanying figures, the present disclosure generally provides a barrier or shield device that is positionable between a patient and at least a portion of a doctor or other person providing health-related services (i.e., a user). The shield device includes a transparent portion such as a viewing screen through which the user can observe the patient while performing a procedure. In one example, the user is a dentist or dental assistant and the procedure is a dental procedure conducted in the patient's mouth.

The transparent portion may be supported by a support assembly. The support assembly may be mounted at a variety of locations such as, for example, a chair or table that the patient is supported on, or the floor, wall, or ceiling adjacent to the patient. The support assembly may be adjustable to adjust the position of the transparent portion. The support assembly may be manually adjustable. In some embodiments, at least some aspects of the support assembly operate automatically to adjust a position of the transparent portion. The support assembly may include multiple arms, joints, and the like. The support assembly may include tensioning members, shock absorbers, motors, sensors, and other electronics. In at least some examples, the support assembly also carries electronics, wiring, tubes, suction or other ventilation devices or components, and the like associated with the shield device.

The transparent portion of the shield device may be referred to as a shield, protector, patient shield, barrier, patient barrier, or the like. The shield may include a frame or base portion, and the frame supports a transparent portion through which the user is able to view a portion of the patient. The frame may carry a number of features and provide various functionality such as, for example, suction ventilation, lighting, displays, user selectable features, speakers, sensors, protecting films, and the like. At least some of these features may be positioned or defined in or on the transparent portion or screen.

Another aspect of the present disclosure relates to confining or controlling the airflow around the patient in association with the shield device. In one example, the shield device includes a skirt or apron that extends from, for example, the frame or perimeter of the shield towards the patient. This apron or skirt provides an area or zone around the patient or a portion of the patient arranged adjacent to the shield. The shield device may provide ventilation such as, for example, a vacuum pressure condition that removes air, moisture, and the like associated with the patient while protecting or limiting the user (e.g., dentist or healthcare provider) from exposure to the patient.

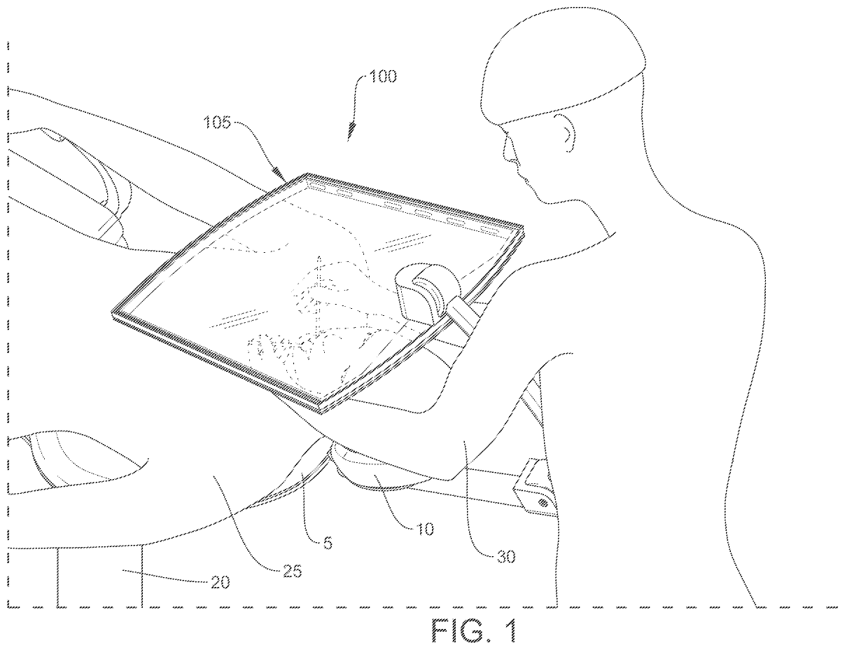

Referring now to FIG. 1, an example shield device 100 is shown in use between a patient 25 and a user 30 (e.g., doctor or dentist). The patient 25 is positioned on a chair 5 having a headrest 10 and a base 20. The shield device 100 may be connected to the patient chair 5 (e.g., to a frame portion of the chair). In alternative embodiments, as described below, the shield device 100 may be connected or supported on other structures such as, for example, a ground surface, a wall, a ceiling, a mobile device or cart, the base 20 of the chair 5, or the user directly.

The shield device 100 includes a shield 105 and a support assembly 110. The shield 105 may include a transparent portion through which the user 30 may view a portion of the patent. In FIG. 1, the user 30 can view the head of the patient through the shield 105 while conducting a procedure on the patient (e.g., a dental procedure in the patient's mouth). The shield device 100 provides a physical barrier between the procedure location on the patent and the face of the user. In the example of a dental procedure, the shield device 100 provides a physical barrier that limits the user's exposure to air being breathed in and out by the patient as well as saliva, water spray, air spray, and contaminated instruments associated with the patient's mouth during the procedure. The shield device 100 may include a variety of features and functionality that provide additional protection of the user from contamination associated with the procedure and/or patient. The shield device 100 may also provide a physical separation or barrier between the patient and the user 30. For example, the shield device 100 may restrict airflow from the user breathing in and out, objects such as eyelashes, hair, saliva or the like from the user contacting the patient's head or area associated with the procedure. Thus, one aspect of the shield device 100 is the ability to protect both the patient and the user from contaminating each other during a procedure.

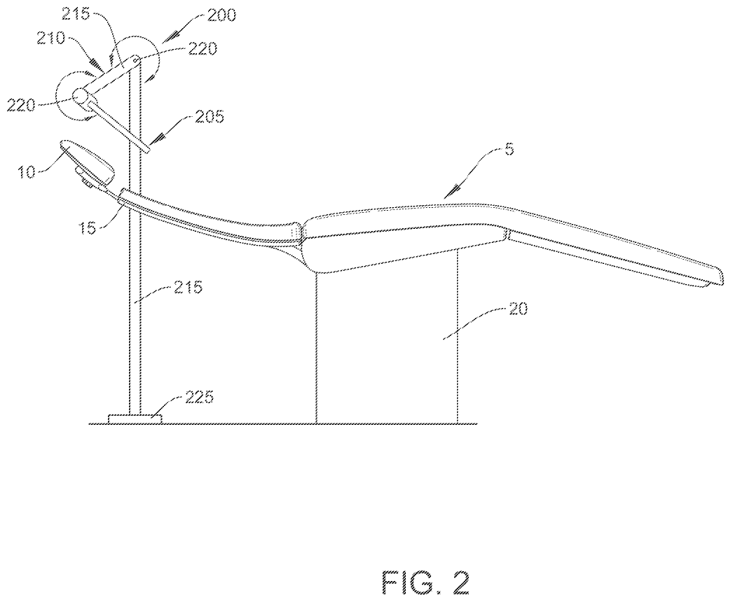

FIG. 2 illustrates another example shield device 200 that is supported on a ground surface. The shield device 200 may be fixed to the ground surface. The shield device 200 may be movably mounted to the ground surface similar to a floor lamp structure. The shield device 200 includes a shield 205 and support assembly 210. The support assembly 210 includes a plurality of arms 215 interconnected with pivot points or axis 220 and supported at a base 225. The arms 215 may rotate relative to each other about the pivot points or axis 220 to position the shield 205 at a desired location relative to the patient. In at least one application, the support assembly 210 may be adjustable to position the shield 205 within a desired distance from a patient's head, and the procedure carried out is a dental procedure on the patient's mouth. The patient may be supported on the chair 5 with the patient's head resting on the headrest 10. The headrest 10 is supported by the frame 15 and base 20.

The support assembly 210 may be adjusted vertically up and down, side to side by moving the base 225 along the ground surface, and through various pivoted positions to obtain the desired orientation for the shield 205. The pivots 220 may provide pivoting in multiple directions such as a multi-axis pivot point (e.g., ball and socket joint). The pivot points 220 may be resistance joints that hold their adjusted position until the user applies a force to move the arms about the pivot points. In any of the examples disclosed herein, the pivot points may include additional features and functionality, including, for example, gas filled struts, biasing members, fasteners, handles or other gripping devices, telescoping features, and the like to provide the desired adjustability position retention, ease of movement, and the like.

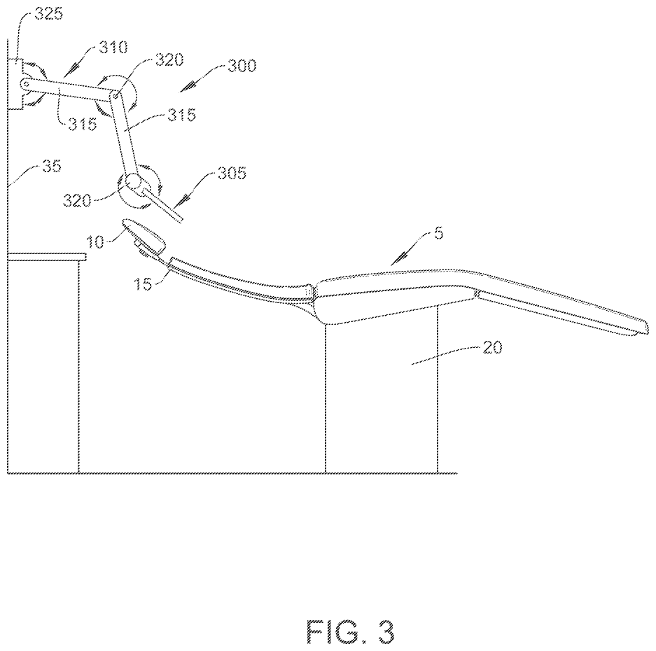

FIG. 3 illustrates another example shield device 300 that is mounted to a wall surface 35. The shield device 300 includes a shield 305 and a support assembly 310. The support assembly 310 includes a plurality of arms 315 interconnected at pivot points 320, and a base 325 that is mounted to the wall 35. The support assembly 310 may include a plurality of adjustment points and other adjustment features such as, telescoping arms and translation of the base 325 along the wall 35, in order to provide the desired positioning of the shield 305 relative to a patient supported on the chair 5.

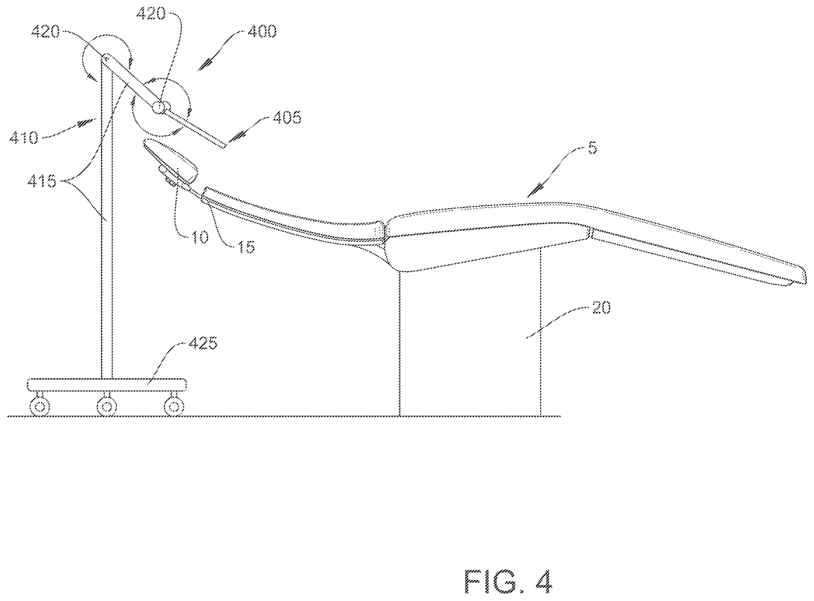

FIG. 4 shows another shield device 400 that includes a shield 405 supported by a support assembly 410. The support assembly 410 is carried on a movable cart or other mobile device. Support assembly 410 includes a plurality of arms 415 interconnected at pivot points 420 and secured to a base 425. The base 425 may include, for example, a plurality of rollers or wheels that provide movement of the shield device 100 along a ground surface. The base 425 may include a brake or other stop feature that helps retain the support assembly 410 in a desired location along the ground surface. The support assembly 410 may be movable into different positions relative to the chair 5. For example, the support assembly 410 may be positioned rearward of the headrest 10 as shown in FIG. 4. Alternatively, the support assembly 410 may be moved such as the base is positioned laterally adjacent to the side surface of the chair 5. A portion of the base 425 may be positioned underneath a portion of the chair 5. The pivot points 420 and arms 415 may be adjustable or function to permit placement of the shield 405 in a desired relative position to the patient regardless of where the base 425 is positioned relative to the chair 5.

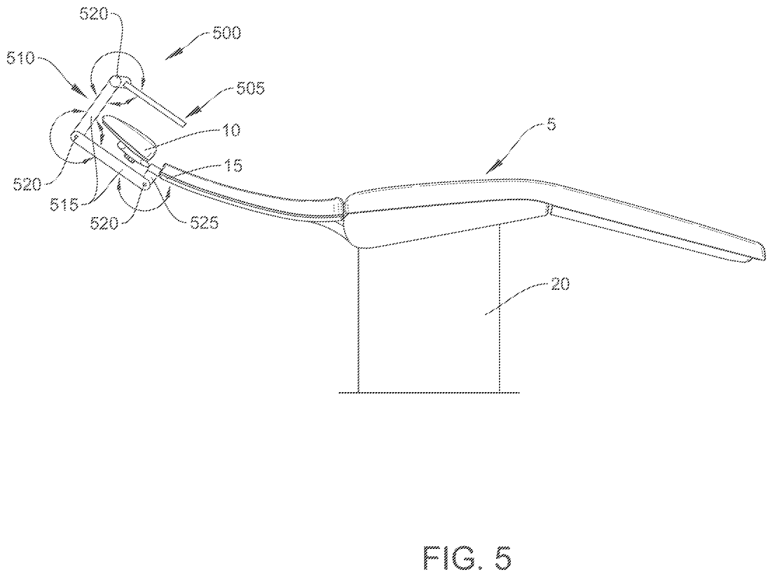

FIG. 5 shows an example shield device 500 that is mounted directly to the chair 5. The shield device 500 includes a shield 505 and support assembly 510. The support assembly 510 includes a plurality of arms 515 interconnected with pivot points 520 and secured to the chair 5 by the base 525. The connection point between the support assembly 510 and the chair 5 may be at any desired location such as, for example, at the frame 15. One mounting point may be to the frame 15 in a location just below the headrest 10, although other locations along the frame 15 are possible.

The support assembly 510 is shown wrapping rearward around the headrest 10 to position the shield directly above the headrest 10. In other examples, the support assembly 510 may be mounted to the frame 15 at the same location shown in FIG. 5, but the arms 515 wrap around a lateral side of the headrest.

The support assembly 510 includes two separate arm members 515 and three pivot points 520. Other examples include a single arm 515 and only two pivot points 520. Yet other examples include more than two arms 515 and more than three pivot points 520. The number of arms and pivot points provide various advantages and disadvantages related to, for example, stability, adjustability, and the like.

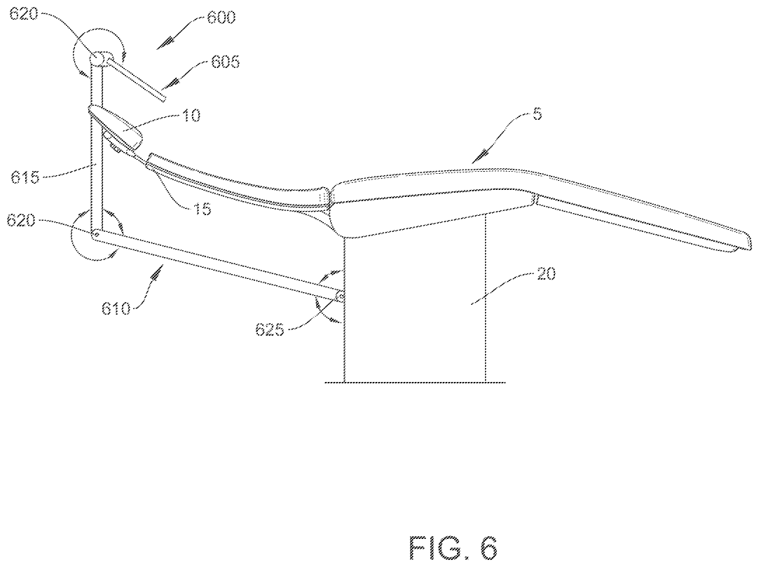

FIG. 6 shows an example shield device 600 that is mounted to the base 20 of the chair 5. The shield device 600 includes a shield 605 and a support assembly 610. The support assembly 610 includes a plurality of arms 615 interconnected with pivot points 620 and mounted to the base 20 of the chair with a base 625. The arms 615 and pivot points 620 may have a variety of adjustment features including, for example, telescoping arms, multi-axis joints, and the like to provide positioning the shield 605 at a desired location relative to a particular feature of the patient (e.g., the patient's head). As shown in FIG. 6, the support assembly 610 may be arranged adjacent to one side or the other of the headrest or other features of the chair 5. Portions of the support assembly 610 may be adjusted to be rearward of the headrest 10. The base 625 may be mounted to the base 620 at a variety of positions such as, for example, on a side of the base 20 closest to the headrest 10. The base 625 may be adjustable vertically and horizontally along the base 625 to provide desired positioning of the shield 605.

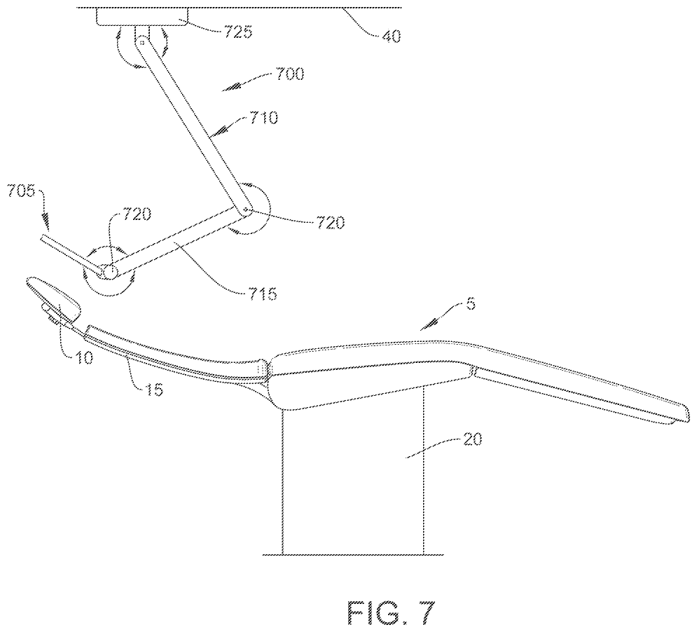

FIG. 7 shows a shield device 700 that is mounted to a ceiling 40. The shield device 700 includes a shield 705 and support assembly 710. The support assembly 710 includes a plurality of arms 715 interconnected with pivot points 720 and mounted to the ceiling 40 with a base 725. The base 725 may be adjustable along the ceiling 40 in a variety of translation directions. The arms 715 and pivot points 720 may provide variety of adjustment features such as telescoping and multi-axis pivoting to position the shield 705 at a desired location relative to any portion of the chair 5. FIG. 7 shows the shield 705 at one orientation relative to the headrest 10 with the shield 705 arranged from the body towards the head of the patient.

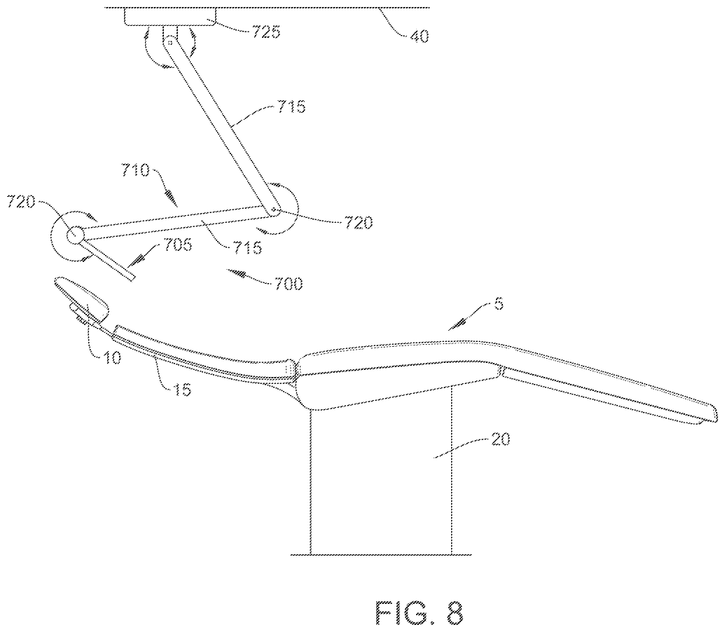

FIG. 8 shows another orientation of the shield device 700 in which the shield 705 is directed from the head towards the body of the patient. In one example, the change in orientation between FIGS. 7 and 8 may be carried out by detaching the shield 705 and flipping it over 180 degrees relative to the support assembly 710. Alternatively, one or more of the pivot points 720 includes a multi-axis joint that permits rotating the shield 705 through 180 degrees between the orientation shown in FIGS. 7 and 8.

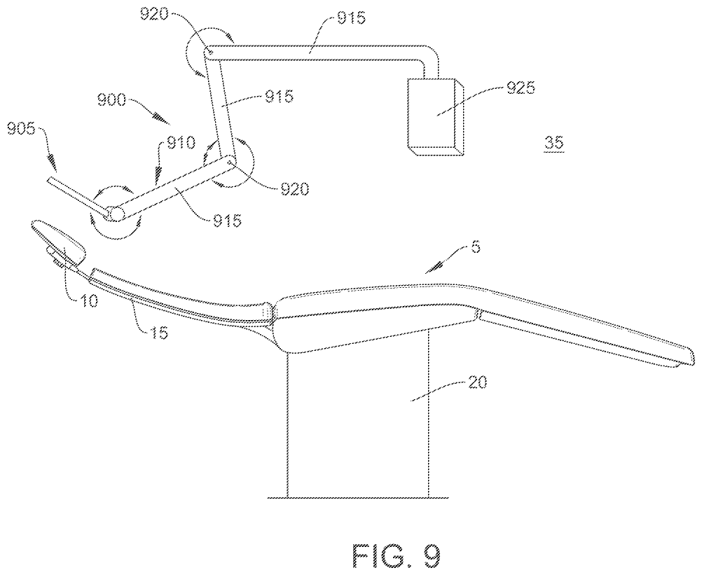

FIG. 9 shows a shield device 900 that is mounted to a wall 35 along a lateral side of the chair 5 as opposed to a wall at the head of the chair that is shown in FIG. 3. Shield device 900 includes a shield 905 and a support assembly 910. The support assembly 910 includes a plurality of arms 915 interconnected with pivot points 920 and mounted to the wall 35 at a base 925. The base 925 may be movable along the wall in vertical and horizontal directions (e.g., along a track). The arms 915 and pivot points 920 may be adjustable in a variety of ways (e.g., telescoping arms, multi-access joints, etc.) to position the shield 905 at a desired location relative to a patient who is supported on the chair 5.

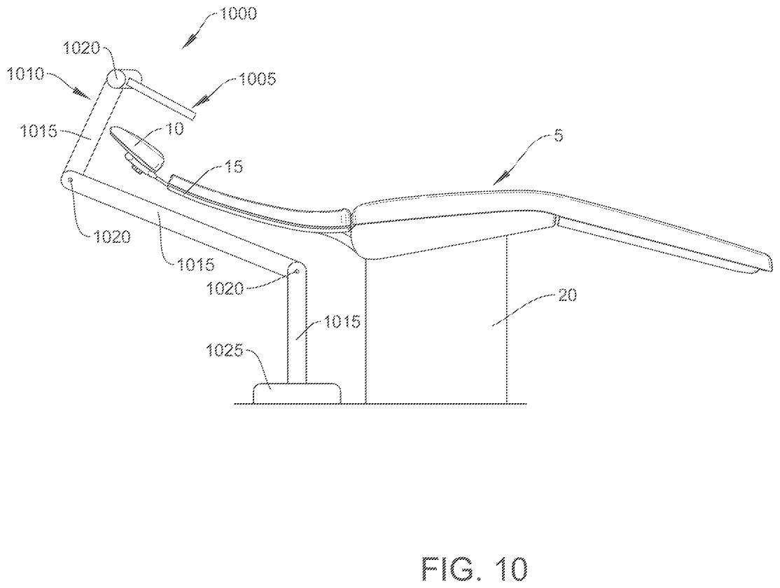

FIG. 10 shows a shield device 1000 that is supported on a ground surface. The shield device 1000 includes a shield 1005 and support assembly 1010. The support assembly 1010 includes a plurality of arms 1015 interconnected with pivot axis 1020, and mounted to a ground surface at a base 1025. The base 1025 may be fixed to the ground surface. The base 1025 may be positioned at any desirable location relative to the chair 5 including, for example, underneath a portion of the chair 5, adjacent to the base 20 and the forward, rearward or lateral direction, etc. The arms 1015 and connection points 1020 may provide a variety of adjustment features, such as telescoping arms and multi-axis joints to provide positioning of the shield 1005 at a desired location relative to a patient supported on the chair 5. In at least some example, certain aspects of the support assembly 1010 are powered. That is, some aspects of the support assembly 1010 operate using power, such as, by one or more motors that move the arms 1015 relative to each other about the pivot points 1020, or adjust the length of one or more of the arms 1015 using an alternative power source besides human power. In at least one example, the support assembly 1010 adjusts the position of the shield 1005 depending on a position of the user's head or other body feature during a procedure carried out on a patient that is supported on a chair 5. In at least one example, a plurality of sensors detect the position and/or other features of the user (e.g., eye position, hand movement, etc.) and then make adjustments such as, for example, to maintain the shield 1005 at an optimum position relative to the location of the procedure on the patient and a particular feature of the user (e.g. the user's head).

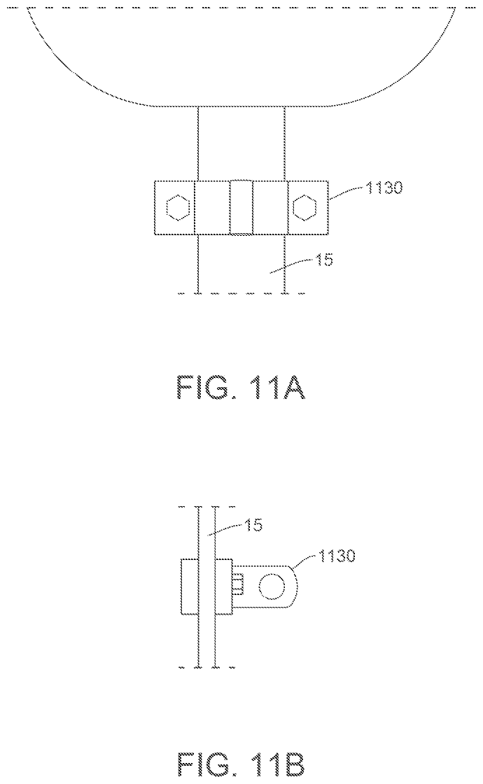

Refer now to FIGS. 11A and 11B, an example mounting bracket 1130 is shown secured to the frame 15 of the chair 5 disclosed with reference to FIGS. 1-10. The bracket 1130 may include a plurality of components that are interconnected to provide a releasable connection to the frame 15. The bracket 1130 may include one or more features for securing the support assembly of the shield device. The bracket 1130 may be connected to or act as the base feature of one or more of the support assemblies disclosed herein.

The support assemblies disclosed with reference to FIGS. 1-10 may contain a plurality of wires, section tubes, pressurized gas lines, data cable, water lines, and other features of the shield device. These components may be carried internal one or more of the arms, base, pivot points, etc. of the support assembly. At least some of these components may be used in association with the shield as will be described below. In other examples, these components may be used a part of carrying out a procedure and may provide a connection point for those components at a location adjacent to the procedure due to the position of the shield and support assembly adjacent to the patient and the user during a procedure.

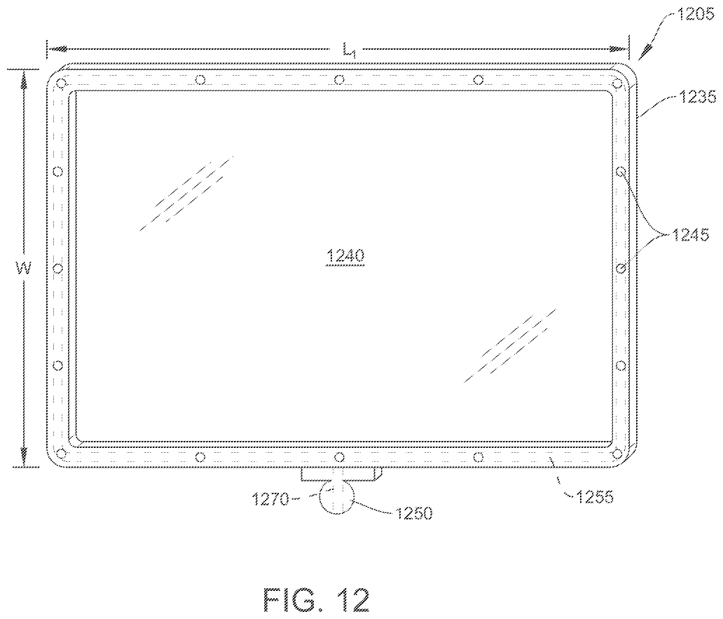

Referring now to FIG. 12, an example shield 1205 is shown and described. The shield 1205 may be an example of any of the shields described above with reference to FIG. 1-10. The shield 1205 includes a frame 1235 and screen 1240. The screen 1240 may include a transparent portion through which a user may view a patient, or a portion of a patient, during a procedure being carried out on a patient. The shield 1205 may have a width W and length L1. The width W and length L1 are each typically in the range of about 6 inches to about 24 inches, and more particularly are in the range of about 12 inches to about 18 inches.

FIG. 12 shows a bottom side 1255 of the shield 1205. The bottom side 1255 is a side that faces the patient. The shield 1205 also includes a connection number 1250. The connection number 1250 may include a connecting feature, such as, for example, a ball feature that fits in a socket of a support assembly. The connection feature may provide adjustability of the shield 1205 relative to the support assembly and/or the patient. The connection number 1250 may include a pass-through channel 1270 through which one or more components may pass from the support assembly to the shield 1205. For example, a pass-through channel 1270 may be receptive of a suction tube, a plurality of wires, communications cables, air supply lines, water supply lines, and the like. These components may pass from the support assembly to the shield 1205 as part of supporting one or more functions or features of the shield 1205, or providing a connection point on the shield 1205 for other devices for use in treating the patient. For example, the frame 1235 may include a port for pressurized air, a port for a pressurized water supply, a port for connecting to a communication line, such as a data wire, a port for power (e.g., low voltage DC or 110 volt AC power), or the like.

FIG. 12 shows a plurality of suction ports 1245 connected to a vacuum suction line. The ports 1245 may be positioned around a perimeter of the screen 1240. The suction ports 1245 may be connected to a section line that is formed internal the frame 1235 and connected to a suction line passing through channel 1270. The suction ports 1245 may provide an air flow that travels from the patient into the shield 1205. The flow into the suction ports 1245 may contain or otherwise prevent flow of the patient's air being breathed in and out or other contaminants associated with the patient from contacting or otherwise traveling to the user or other people surrounding the patient during a procedure.

The frame 1235 may comprise any of a variety of materials such as, for example, Aluminum, polymer materials, or composite materials, and may have many different properties such as, for example, electrically conductive, insulating, or anti-bacterial. The screen 1240 may comprise of various materials that are transparent or translucent, such as, for example, acrylic material, tempered glass, LCD screens, or the like. The screen 1240 may comprise a disposable cover or layer, such as a protective film, that is removably mounted to the screen 1240 or frame 1230. The protective film or cover may be replaced for use with each patient. The protective film or cover may be positioned on either side of the shield 1205, or may cover substantially all of the shield.

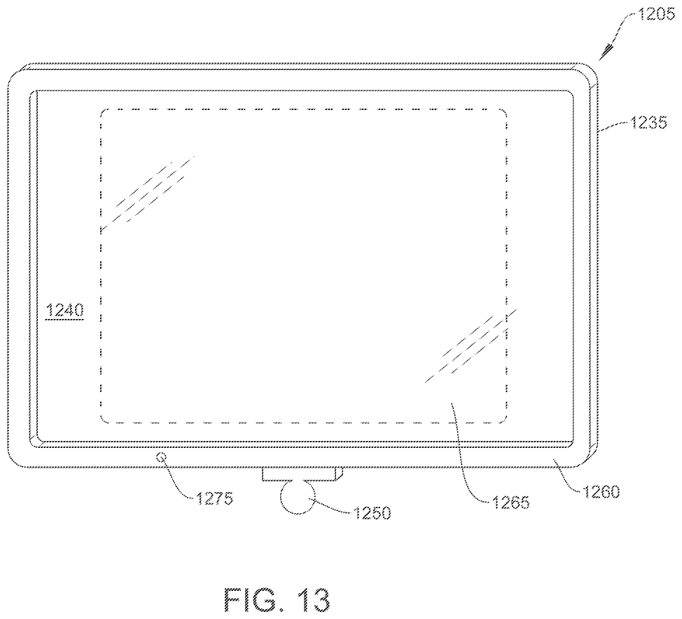

FIG. 13 shows a top side 1260 of the shield 1205. The frame 1235 and screen 1240 are also exposed and viewable along the top side 1260. The screen 1240 may include a magnification portion 1265. The magnification portion 1265 may include an adjustable magnification such as a 2× or 4× magnification capability. In some examples, different sections or portions of the screen 1240 may include different magnification such that the user can look through different portions of the screen to get a different magnification associated with viewing the procedure or feature of the patient.

The shield 1205 may include a camera 1275. The camera 1275 may be positioned or otherwise mounted to the frame 1235. The camera 1275 may be capable of taking still shots and/or video. The camera 1275 may be directed to the user, so as to take pictures or videos of the user, such as a narrative associated with conducting the procedure. Such documentation associated with the procedure may be necessary for insurance records, medical records, and the like. As will be described below, a separate camera may be mounted to the bottom side 1255 of the shield 1205 to document aspects of the procedure looking at the patient and/or location of the procedure.

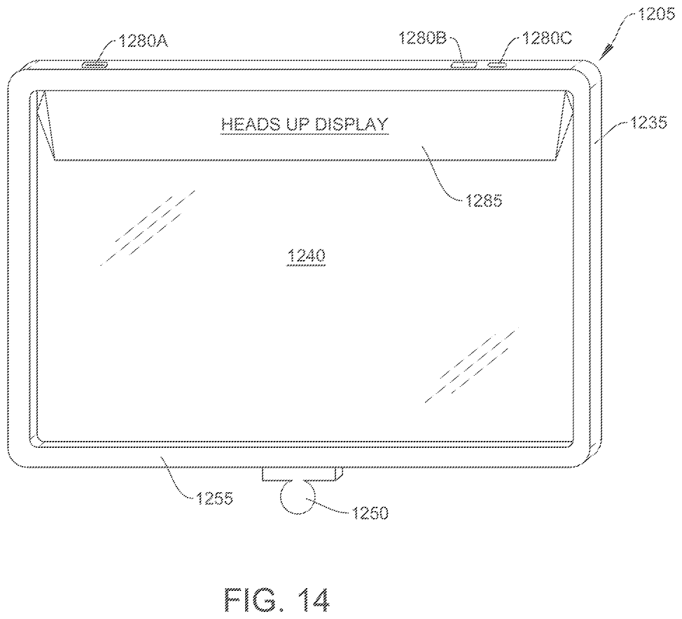

FIG. 14 shows a display 1285 on the shield 1205. The display 1285 may be positioned along one of the edges of the screen 1240 and/or frame 1235. The display 1285 may be arranged at an angle relative to the screen 1240 such as, for example, at an angle in the range of about 30 degrees to about 60 degrees relative to the surface of the screen 1240. The display 1285 may display different types of information associated with the patient and/or the procedure, or provide communications for the user unrelated to the patient (e.g., email and intercom). For example, a portion of the display includes a visual indicator showing physical properties and conditions of the patient such as O2 levels, CO2 levels, EKG reading, heart rate, body temperature, and the like. Another portion of the display may show information from the patient's medical, dental, or other history, an x-ray of a portion of the patient (e.g. the patient's teeth), an audio or visual indicator associated with an intercom for the doctor's office or hospital, the time of day, or the like. The display may be connected to a wired connection or through a wireless connection. Various wireless connections include Wi-Fi, Bluetooth, or the like. Other wired connections include CAT 6, 7 data, HDMI, Chromecast/Miracast, O2, CO2, EKG connections direct, a phone line, or the like. The display may be connected to wires passing through the frame 1235 and the channel 1270 out of the connector 1250.

A variety of connection ports may be provided in the frame 1235 such as, for example, a phone charger port 1280A, a USB port 1280B, a USB-C port 1280C, or other communications ports.

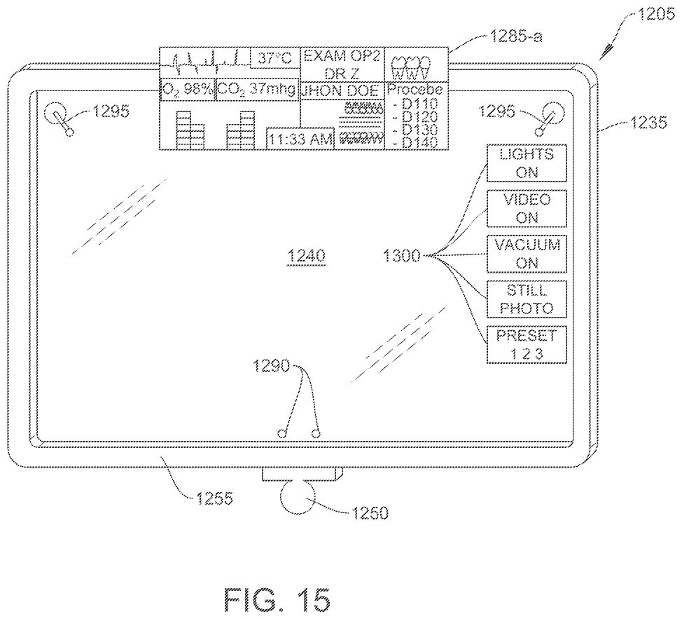

FIG. 15 shows the shield 1205 having a display 1285-a. The display 1285-a includes different section providing different types of information, such as the information described above with reference to display 1285. In a dental application, the display 1285-a may show patient dental x-rays, O2 and CO2 readings, EKG readings, patient history, doctor information, and time of day. Thus, display 1285-a may provide relevant information associated with the patient directly in the user's (e.g. dentist's) line of view during a procedure, so the user does not have to look away to a different screen or location to gather than information during the procedure.

The shield 1205 may also include a plurality of joy sticks 1295 associated with light sources pointing towards the patient. The light sources may include, for example, one or more LED lights that can be directed to the area of the procedure (e.g., patient's mouth). While two joysticks 1295 associated with two light sources are shown, other arrangements include different numbers of light sources, such as three or more light sources or a single light source. Some of the light sources may be fixed while other light sources are adjustable. The lights sources and adjustment features may be mounted to the screen 1240 or may be mounted to the frame 1235.

The shield 1205 may include a plurality of user selectable options 1300. The options 1300 may be spaces defined along the screen 1240 such as touch enabled areas or zones on the screen 1240. The selectable options may include, for example, on/off features or other controls for various functionality associated with the shield 1205. Some example controls associated with the touch buttons 1300 include turning the lights on and off, turning the video on and off, turning the vacuum on and off, taking still photos, turning video on and off, or selecting among various preset settings for the shield 1205 (e.g., preset settings for the lights, camera, suction, or other features).

The shield 1205 may also include a plurality of sensors 1290 that are directed to the user. The sensors 1290 may include, for example, eye tracking sensors that track the location and/or movement of the user's eyes during the procedure. The shield device may automatically respond to movement of the eyes. Such automatic adjustments may include, for example, adjusting the physical position of the shield 1205 relative to the user's head and/or relative to the location of the procedure. In one example, the sensors 1290 provide feedback regarding the position of the user's head to maintain alignment of the shield 1205 in a line of sight between the user's eyes and the location of the procedure (e.g., the patient's mouth during a dental procedure). Other types of sensors besides eye tracking sensors may be used including, for example, motion sensors, and the like. Various numbers of sensors may be used at different locations on the shield 1205, such as, for example, at different locations along the frame 1235 or at corners of the screen 1240. The sensors may be coupled to a robotic arm or similar device that is associated with the support assembly of the shield device. The robotic arm may move the entire shield device or a portions thereof automatically based on feedback from the sensors 1293.

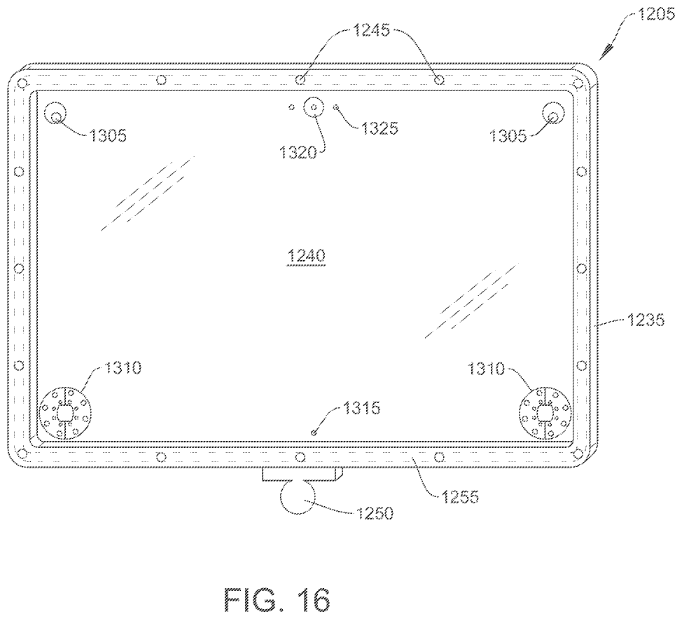

FIG. 16 shows the bottom side 1255 of the shield 1205 with additional features on the frame 1235 and screen 1240. For example, the shield 1205 may include, in addition to the plurality of suction ports 1245, one or more light sources 1305 on the frame 1235 or screen 1240. The light sources 1305 may include, for example, one or more LED lights. The light sources 1305 may include a lens that is adjustable to focus a beam of light on the location of the procedure being conducted. The light sources may be adjustable from an opposite top side 1260 of the shield 1205 using, for example, the joy sticks 1295 described above. A single light source or three or more light sources may be positioned along the bottom side 1255 to illuminate the patient, the site of the procedure, or other features such as the instruments being used during the procedure.

Shield 1205 may also include speakers 1310. The speakers may be directed towards the patient so that the patient can listen to music or other media during the procedure. The speakers 1310 may also be used to convey instructions for commentary from the user that may otherwise be difficult to hear because the shield 1205 is interposed between the user and the patient. A microphone may be positioned on the top side 1260 to pick up the voice of the user, and the speakers 1310 project that sound to the patient. The speakers 1310 may be positioned on the frame 1235. A single speaker or three more speakers may be used. The shield 1205 may include an auxiliary port to connect headphones. Alternatively, the shield 1205 may include Bluetooth to connect to wireless headphones or other wireless media worn by the patient and the shield 1205 may be connected wirelessly to other remote located devices such as a network of the office, etc.

Shield 1205 may include a bottom camera 1320 that can take still shots and/or video of the patient. One or more microphones 1325 may also be positioned to collect audio from the patient. A tracker (e.g., laser tracker) may be positioned along the bottom side 1255 to auto adjust the lights 1305 or other features of the screen 1205. A variety of other sensors and electronic devices and functionality may be positioned on the bottom side 1255 to assist in interacting with the patient, operating and/or monitoring features and functionality of the screen 1240, or collecting data about the environment between the shield 1205 and the patient. For example, a patient temperature sensor 1315 may be arranged on the bottom side 1255 to monitor the patient's temperature either directly or via a remote sensing device. Sensor 1315 may collect other information such as, for example, oxygen levels, presence of contaminants in the air, air flow, and the like.

Other types of filters may be used on the screen, including a laser filter for filtering light from CO2 lasers, diode lasers, or yttrium aluminum garnet (YAG) lasers.

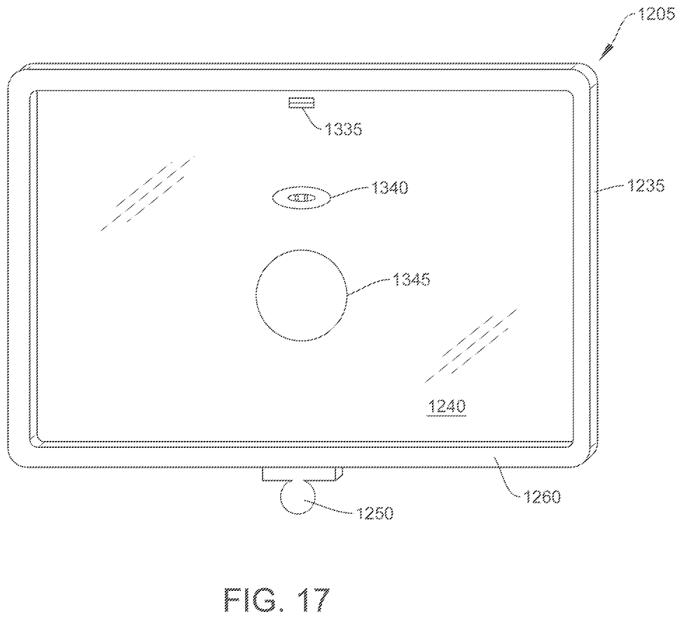

FIG. 17 shows the screen 1205 with a jack or port 1335 positioned on the screen at 1240. The jack or port 1335 may alternatively be positioned on the frame 1235. The jack or port 1335 may be used to connect the computing device such as, for example, a hand-held mobile device (e.g., iPhone, iPad, Galaxy, or the like). The port or jack 1335 may be used to physically secure the mobile computing device to the shield 1205 and may also concurrently provide electronic connection with the mobile computing device. The mobile computing device, when connected to the port or jack 1335, may act as the display 1285. The mobile computing device may operate an application that duplicates the features and functionality of the displays 1285 described above. The mobile computing device may provide additional functionality including, for example, texting, emails, additional mobile applications, internet connections, and the like.

FIG. 17 also illustrates a microscope mount 1340 for mounting a microscope to the shield 1205. The shield 1205 may include a microscope opening 1345 formed in the screen 1240. The microscope may be mounted to the microscope mount 1340 and the lens of the microscope may be aligned with the opening 1345. A portion of the microscope may fill the opening 1345 and/or form a seal with the perimeter of opening 1345 so as to maintain the barrier between the patient and the user. The microscope mount 1340 may be a quick mount structure. The opening 1345 may have any desired shape and size to accommodate a particular microscope being used. In at least some embodiments, a microscope may be mounted to the mount 1340 and operable to perform its intended purpose without the opening 1345 in the screen 1240.

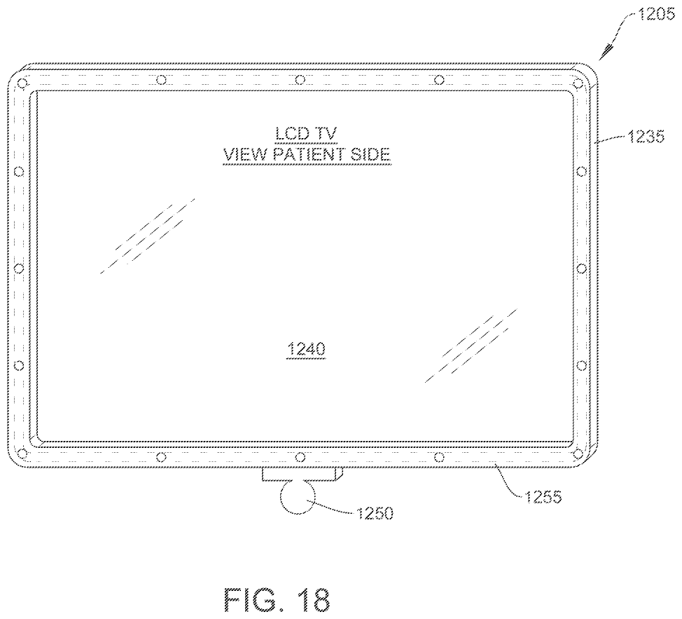

In at least some examples, the screen 1240 may act as an LCD or other type of monitor screen for viewing by the patient along the bottom side 1255, as shown in FIG. 18. The patient may be able to view media or other content on the bottom side of the screen 1240, while the screen still provides a transparent portion for viewing through by the user to see the treatment site. In at least some examples, only a portion of the screen 1240 is dedicated to serving as a display or monitor for viewing media by the patient.

The patient viewable screen may also be used to permit the patient to view the procedure via the patient facing camera that is recording the event. The LCD screen may also permit the patient to view educational videos, read instructions, and the like, associated with the procedure, recovery from the procedure, and best practices, and the like, that the patient should follow or understand, based on the treatment and/or procedure. The patient monitor or screen may be connected to the internet, social media sites, video conferencing, or the like, to permit the patient to be connected with remotely located people prior to, during, or after the procedure.

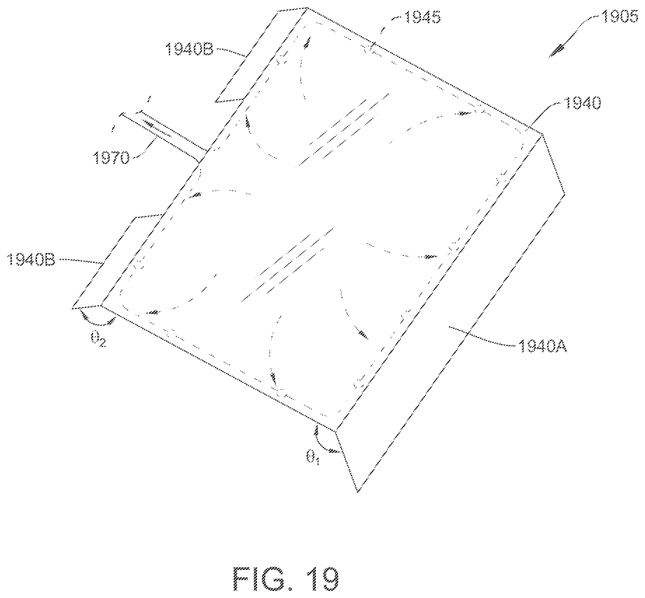

FIG. 19 illustrates another example shield 1905 that includes a screen 1940 and one or more flaps or barrier members 1940A, 1940B. The shield 1905 may include internal channels and a plurality of suction ports 1945 that create a negative air flow condition. This negative air flow condition may draw air away from the patient and the user and into a suction line 1970. The shield 1905 may include internal channels, also referred to as ventilation channels. The ventilation channels may be positioned along an exterior surface of the shield 1905, such as, for example, along the screen 1940, along a frame, along a top surface (facing the user), or bottom surface (facing the patient) of the shield 1905. The ventilation ports 1945 may be positioned along the top or bottom side of the shield 1905 to draw air away from the user and/or the patient. The flaps or shields 1940A, 1940B may provide additional separation between the user and the patient. The flaps 1940A, 1940B may be positioned at an angle θ1, θ2 relative to the screen 1940. The flaps 1940A, 1940B may be positioned along opposite side edges of the screen 1940, along a single edge, or along all edges of the screen 1940.

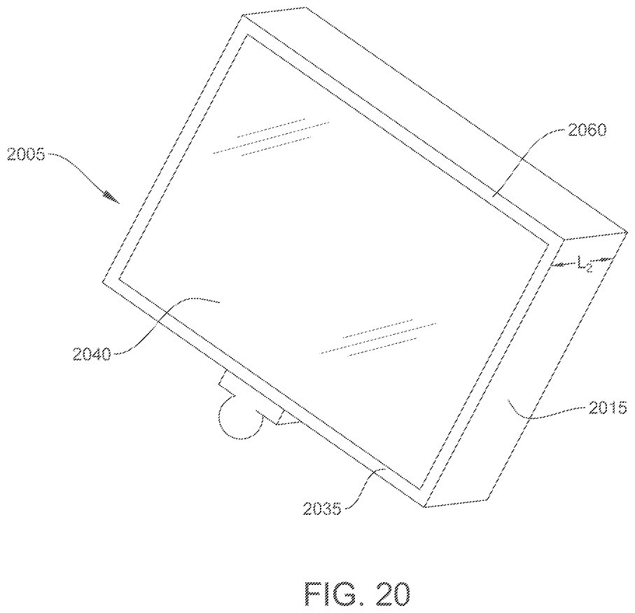

FIG. 20 shows another example shield 2005 having a frame 2035 and a screen 2040. The skirt or curtain 2015 may extend from the shield 2005 towards the patient (i.e., in a direction away from or an opposite direction from the top surface 2060). The skirt 2015 may have a length L2 extending from the top surface 2060 towards the patient. The length L2 may be in the range of about 2 inches to about 10 inches, and more particularly about 4 inches to about 6 inches. The skirt 2015 may comprise of a variety of different materials. The skirt 2015 may comprise a disposable material that is intended to be replaced after each procedure and/or after use with a given patient. The skirt 2015 may comprise a rigid material so as to maintain its shape even when exposed to the negative pressure condition associated air flow when a ventilation system is in use along the bottom side of the shield 2005.

The skirt 2015 may comprise a relatively soft material that promotes user comfort in the event that the skirt 2015 contacts the patient. In some examples, the skirt 2015 comprises a reusable material, such as a material that can be washed or sanitized after use. In at least some examples, the skirt 2015 is releasably connected to the shield 2005. A variety of connection features and methods may be used to secure the skirt 2015 to the shield 2005. For example, an elastic border, hook and loop fastener material, snaps, zippers, brackets, rigid frames, or the like may be used to connect the skirt 2015 to the shield 2005. The skirt 2015 may be connected to the screen or frame portion of the shield 2005. Typically, the skirt provides an airtight connection or is secured to the shield 2005 with an airtight connection.

The space or zone defined between the bottom side of the shield 2005 and the inward facing surface of the skirt 2015 may be defined as a containment area. The containment area is intended to contain not only air, but other contaminants such as saliva, spraying water, blood, and the like that may result from a procedure being conducted. Those materials so forth in the containment area are contained relative to the patient and contained with respect to contact by the user.

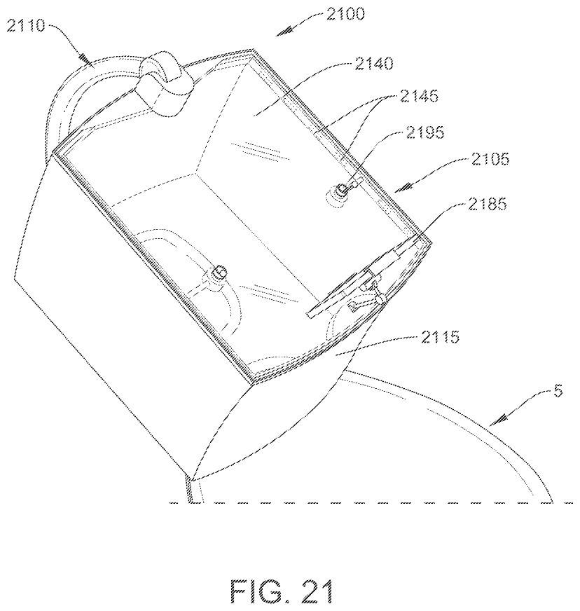

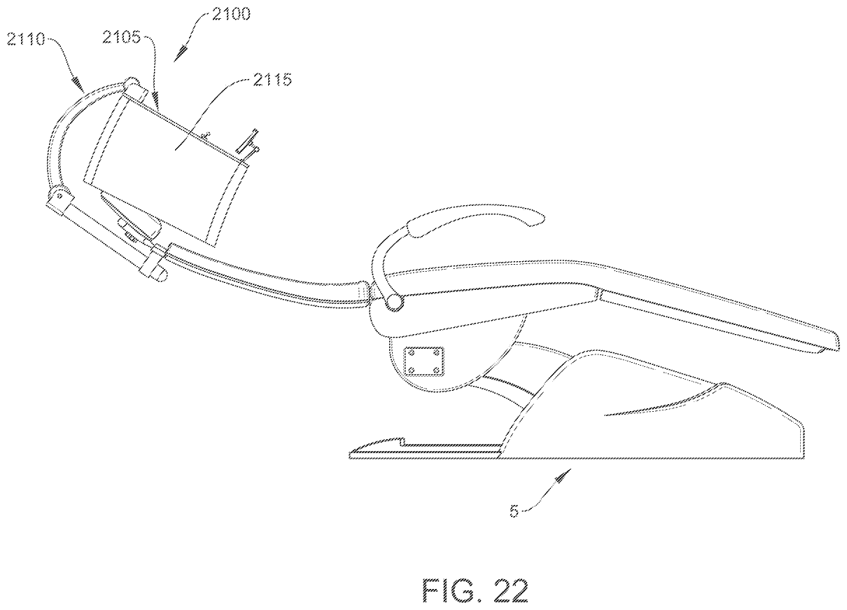

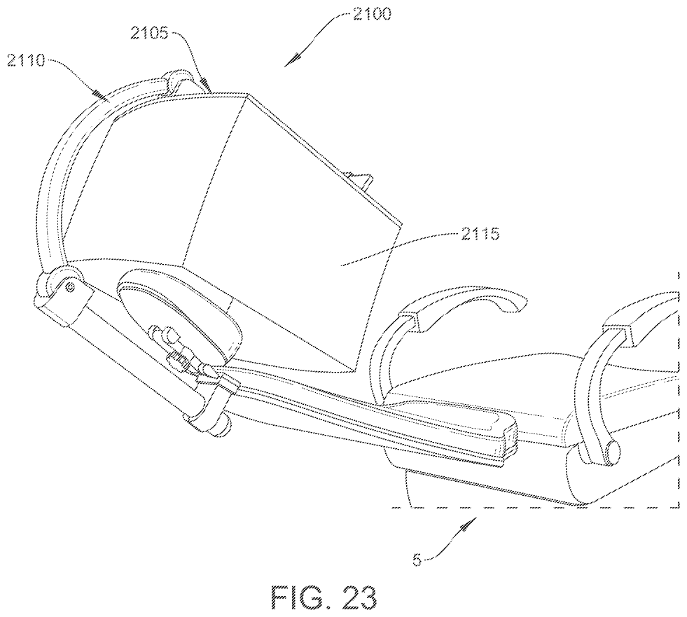

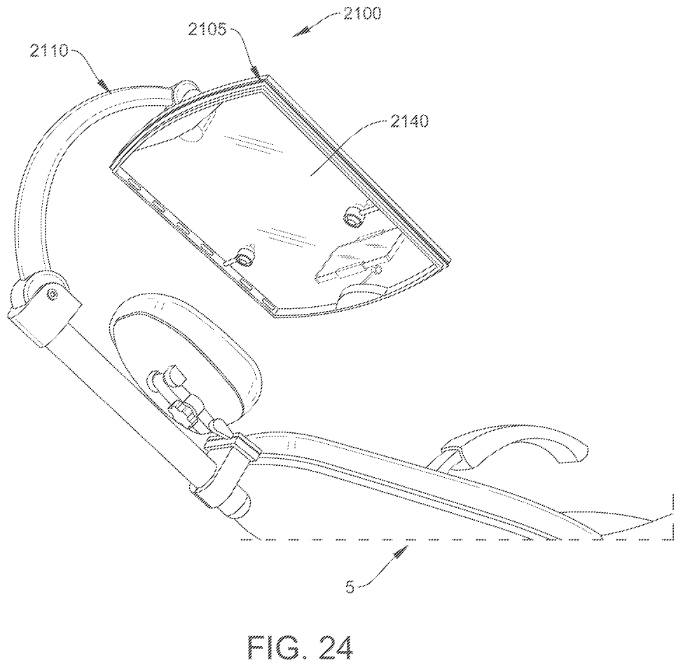

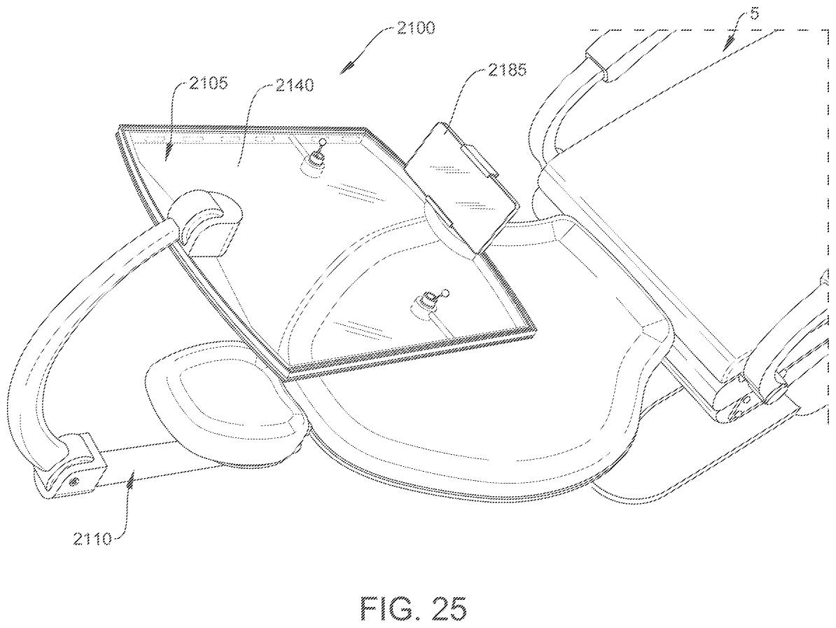

FIGS. 21-25 illustrate another example shield device 2100. The shield device 2100 includes a shield 2105 and support assembly 2110. The support assembly 2110 is mounted directly to a chair 5. The shield device 2100 includes a skirt 2115 that is mounted to the shield 2105. Shield 2105 includes a variety of features, including, for example, a screen 2140, lights 2195 and a display 2185. A skirt 2115 is shown as a fabric material, but may be any material that meets the criteria set forth above. The shield 2105 may also include a plurality of ventilation ports or suction ports 2145 as shown in at least FIG. 21. The suction ports 2145 may be connected to a vacuum line and may create an air flow from within the containment space defined between the bottom side of the shield 2105 and the inner surface of the skirt 2115.

Although the support assembly 2110 is shown mounted directly to a frame of a chair 5, the support assembly may be mounted to other objects as described above with reference to FIGS. 1-10. The support assembly 2110 may be operable to move the shield 2105 between an active position, as shown in FIGS. 21-25, arranged adjacent to the patient, so as to be interposed between the user and the patient, and an inactive or removed position, such that the shield 2105 is not positioned between the user and the patient (and/or procedure site). The support assembly 2110 may move the shield 2105 laterally, vertically, through rotation, or in a variety of different ranges of motion to move between the active and inactive positions. The support assembly 2110 may be actuated manually by the user or another person. Alternatively, the support assembly 2110 may automatically operate to adjust the position of the shield 2105 based on, for example, sensor feedback and powered adjustment features of the support assembly.

The shield device 2100 may be used with or without the skirt 2115 shown in FIGS. 21-23. The shield device 2100 may include any of the features or functionality of the various embodiments disclosed herein with reference to the remaining figures.

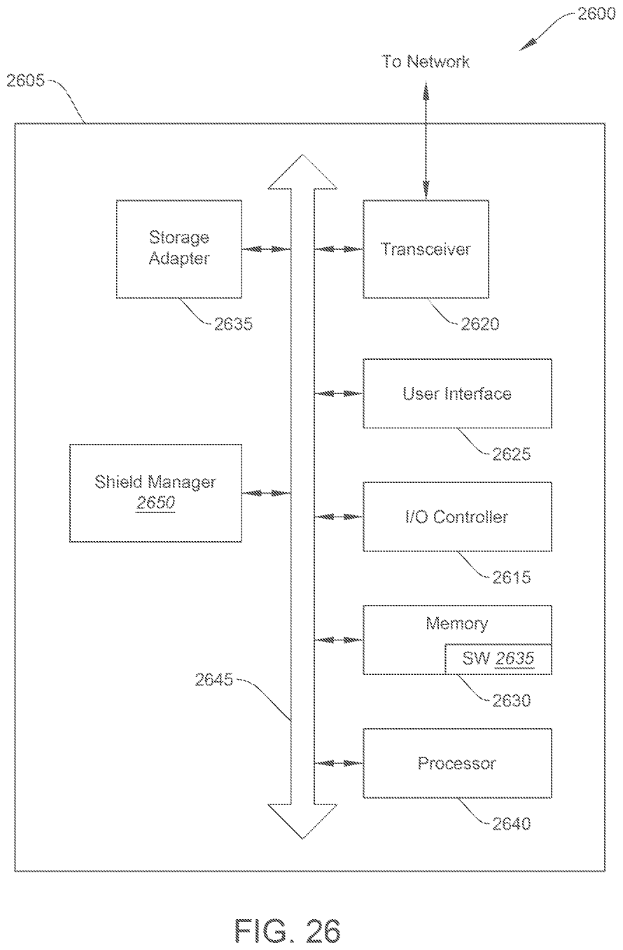

FIG. 26 shows a diagram of a system 2600 including a device 2605 that supports a shield device in accordance with aspects of the present disclosure. The device 2605 may be an example of or include the components of any of the shield devices, shields, or shield displays described herein. The device 2605 may include components for data communications including components for transmitting and receiving communications, including a shield manager 2650, a storage adapter 2610, an I/O controller 2615, a transceiver 2620, a user interface 2625, memory 2630, and a processor 2640. These components may be in electronic communication via one or more buses (e.g., bus 2645).

The shield manager 2650 may provide control of one or more aspects of the shield devices disclosed herein. For example, the shield manager may receive user inputs, process instructions, communicate data to a user, display content, and the like. Some of the operations facilitated by shield manager 2650 may conducted automatically in response to stored instructions, code, or algorithms. The shield manager 2650 may communicate wirelessly with a server via a network, wherein data transferred via the network is stored in one or more databases. Aspects of the shield manager 2650 may operate on site, such as being integral with one or more operational features of the shield devices disclosed herein. In other examples, aspects of the shield manager 2650 are stored and/or operational using a local or remotely located service or network, or stored and/or operation using the remotely stored database.

The I/O controller 2615 may manage input and output signals for the device 2605. The I/O controller 2615 may also manage peripherals not integrated into the device 2605. In some cases, the I/O controller 2615 may represent a physical connection or port to an external peripheral. In some cases, the I/O controller 2615 may utilize an operating system such as iOS®, ANDROID®, MS-DOS®, MS-WINDOWS®, OS/2®, UNIX®, LINUX®, or another known operating system. In other cases, the I/O controller 2615 may represent or interact with a modem, a keyboard, a mouse, a touchscreen, or a similar device. In some cases, the I/O controller 2615 may be implemented as part of a processor. In some cases, a user may interact with the device 2605 via the I/O controller 2615 or via hardware components controlled by the I/O controller 2615.

The transceiver 2620 may communicate bi-directionally, via one or more antennas, wired, or wireless links as described herein. For example, the transceiver 2620 may represent a wireless transceiver and may communicate bi-directionally with another wireless transceiver. The transceiver 2620 may also include a modem to modulate the packets and provide the modulated packets to the antennas for transmission, and to demodulate packets received from the antennas.

The memory 2630 may include RAM and ROM. The memory 2630 may store computer-readable, computer-executable code 2635 including instructions that, when executed, cause the processor to perform various functions described herein. In some cases, the memory 2630 may contain, among other things, a BIOS which may control basic hardware or software operation such as the interaction with peripheral components or devices.

The processor 2640 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 2640 may be configured to operate a memory array using a memory controller. In other cases, a memory controller may be integrated into the processor 2640. The processor 2640 may be configured to execute computer-readable instructions stored in a memory (e.g., the memory 2630) to cause the device 2605 to perform various functions (e.g., functions or tasks supporting a shield device).

The code 2635 may include instructions to implement aspects of the present disclosure, including instructions to support a shield device. The code 2635 may be stored in a non-transitory computer-readable medium such as system memory or other type of memory. In some cases, the code 2635 may not be directly executable by the processor 2640 but may cause a computer (e.g., when compiled and executed) to perform functions described herein.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the present systems and methods and their practical applications, to thereby enable others skilled in the art to best utilize the present systems and methods and various embodiments with various modifications as may be suited to the particular use contemplated.

Unless otherwise noted, the terms “a” or “an,” as used in the specification and claims, are to be construed as meaning “at least one of.” In addition, for ease of use, the words “including” and “having,” as used in the specification and claims, are interchangeable with and have the same meaning as the word “comprising.” In addition, the term “based on” as used in the specification and the claims is to be construed as meaning “based at least upon.”

What is claimed is:

1. A shield device for use with a dental chair, comprising:
a support arm;
a shield mounted to the support arm and movable between an active position vertically above a patient's head when the patient is lying in the dental chair, and an inactive position removed from above the patient's head, the shield having a transparent portion through which the patient's head is viewable when the shield is in the active position, the shield including a ventilation system to remove air from a space defined between the patient's head and the shield when the shield is in the active position.

2. The shield device of claim 1, wherein the ventilation system includes at least one vent opening positioned on a side of the shield facing the patient.

3. The shield device of claim 1, wherein the shield includes a frame member surrounding the transparent portion, and the ventilation system includes a plurality of vent openings formed in the frame member on a side of the shield facing the patient.

4. The shield device of claim 1, further comprising a skirt connected to the shield and extending toward the patient.

5. The shield device of claim 1, further comprising at least one of a light source mounted to the shield and directed toward the patient, a speaker mounted to the shield, a display mounted to the shield and directed toward the patient for viewing by the patient, and at least one camera mounted to the shield, the at least one camera directed toward the patient.

6. The shield device of claim 1, wherein the shield includes a touch screen configured to receive touch inputs from a user.

7. The shield device of claim 1, wherein the shield includes a magnification portion, or a portion that have an adjustable magnification feature.

8. The shield device of claim 1, further comprising a user display mounted to the shield and configured to display at least one visual indicator or message.

9. The shield device of claim 10, wherein the user display displays patient vital signs in real-time.

10. The shield device of claim 10, wherein the user display displays at least one of a patient's dental history, x-rays of the patient's teeth, current time, and recommended instructions for a dental procedure.

11. The shield device of claim 1, further comprising at least one communications port, the at least one communications port configured to connect a mobile communications device to the shield.

12. The shield device of claim 1, further comprising a disposable cover extending over at least one surface of the shield.

13. The shield device of claim 1, wherein the transparent portion of the shield includes at least one of tempered glass and acrylic material.

14. The shield device of claim 1, further comprising at least one sensor to detect one or more of temperature, light, eye position of the user, sound frequency, sound volume, smoke, oxygen saturation, and chemical compounds.

15. The shield device of claim 1, wherein the support arm is mountable to at least one of a chair in which the patient is sitting, a ceiling at a location vertically above the patient, a wall positioned adjacent to the patient, and a floor or supported by a floor.

16. The shield device of claim 1, wherein the support arm includes at least one of a plurality of arm segments to adjustably position the shield at a desired location relative to the patient, a plurality of pivot joints coupling together the plurality of arm segments, or a controller to move the plurality of arm segments automatically in response to user controls, the user controls including at least one of a touch input or an audible input.

17. The shield device of claim 1, further comprising a laser filter mounted to the shield and configured to filter at least one of CO₂, Diode, and yttrium aluminum garnet (YAG) laser light.

18. The shield device of claim 1, wherein a portion of the ventilation system extends within or along the support arm.

19. The shield device of claim 1, further comprising a microscope mount configured to releaseably mount a microscope for viewing the patient or procedure site.

20. A shield device for use with a dental chair, comprising:
a support arm;
a shield mounted to the support arm and configured to be positioned above a patient's head when the patient is lying in the dental chair, the shield comprising:
a frame to which the support arm is mounted;
a transparent portion mounted to the frame, the patient's head being viewable through the transparent portion;
a ventilation system configured to remove air from a space defined between the patient's head and the shield, at least a portion of the ventilation system being defined by or supported by the frame.
